(19) United States
(12) Patent Application Publication
(10) Pub. No.: US 2017/0103183 A1
(43) Pub. Date: Apr. 13, 2017

(54) Model Generation for Drug Determination

(71) Applicant: NORWEGIAN UNIVERSITY OF SCIENCE AND TECHNOLOGY, Trondheim (NO)

(72) Inventor: Åsmund Flobak, Trondheim (NO)

(21) Appl. No.: 15/291,341

(22) Filed: Oct. 12, 2016

(30) Foreign Application Priority Data
Oct. 13, 2015 (GB) 1518078.9

(51) Int. Cl. G06F 19/00; A61B 5/00

(52) U.S. Cl. CPC G06F 19/3437; A61B 5/4839; A61B 5/4848

(57) ABSTRACT

Disclosed herein is a computer-implemented method for simulating a response of a drug, or a combination of drugs, being used for the treatment of a disease, the method comprising a computing device performing the steps of: generating one or more models of cell responses in a biological network of cellular processes, wherein each model is a self-contained logical model that comprises a network topology with nodes, edges between nodes and parameters of the nodes for modelling obtained state data of a plurality of biological signalling entities of one or more diseased cells, wherein generating each model comprises automatically determining logical rules that define at least the parameters of the nodes such that an attractor of the model substantially corresponds to said obtained state data of the plurality of biological signalling entities of one or more diseased cells; for each of the one or more models, simulating the effect of a drug, or a combination of drugs, by determining an output of the generated model when the states of one or more nodes of the model are changed in accordance with the expected effect of the drug, or the combination of drugs, on one or more of said biological signalling entities; and determining a drug, or a combination of drugs, for the treatment of the one or more diseased cells in dependence on the outputs of the one or more models. Advantageously, a fast and inexpensive technique is provided for determining drug(s), or combinations of drugs, for the treatment of the disease.

### FIELD

The present invention relates to the generation and use of one or more models for aiding the determination of drug(s) for the treatment of a disease. At least one model of signalling states of one or more diseased calls is generated. The at least one model is a self-contained state based logical model that has an attractor that substantially corresponds to experimentally determined steady states of signalling entities of the one or more diseased cells. The at least one model is used to simulate the effect of a drug, or a combination of drugs, on the one or more diseased cells. Advantageously, a fast and inexpensive technique is provided for determining drug(s), or combinations of drugs, for the treatment of the disease.

### BACKGROUND

The development of novel anti-cancer medication predominantly focuses on drugs directed against specific molecular targets. However, clinical applications have often been disappointing, resulting only in transient responses followed by drug resistance which hinders therapy benefits. This has led to the consideration of therapies based on combinations of drugs targeting different signalling pathways or cellular processes, with the aim to restrain the evolution of drug resistance and at the same time allow for a reduction in drug dosage, to lower drug-induced toxic effects.

The effectiveness of combinatorial anti-cancer treatments can be improved by exploiting synergistic drug actions, meaning that different drugs administered together exhibit a potentiated effect compared to the individual drugs. Drug synergy is attractive because it allows for a significant reduction in the dosage of the individual drugs, while retaining the desired effect. Synergies therefore hold the potential to increase treatment efficacy without pushing single drug doses to levels where they lead to adverse reactions.

These incentives for combinatorial drug treatment are challenged by the numerous combinations to consider. In addition, the determination of an effective drug, or an effective combination of drugs, is complicated by the efficacy of the drug, or drug combination, being dependent on the specific nature of a tumor. Due to the large number or drugs and tumor variants, it remains a tremendous challenge to identify efficient combinations in preclinical pipelines, and even more so in patient setting. To illustrate this, a relatively small set of 150 drugs corresponds to more than 10000 possible pairwise drug combinations. Experimental testing of all possibilities is clearly impossible. Add to this the heterogeneity among cancer cell lines and patient tumors, and the experimental search space that must be covered is vast.

Known techniques for identifying beneficial combinatorial anti-cancer therapies typically rely on large-scale experimental perturbation data, either for deciding on specific patient treatment, or for pre-clinical pipelines to suggest new drug combinations. However, these known techniques face challenges posed by the large search space that needs to be supported by experimental data, making systematic searches for efficient combinations challenging. Moreover, the number of conditions for testing dramatically increases when considering higher-order combinations, multiple drug dosages, temporal optimization of drug administration, and diversity of cancer cell types and patients.

Accordingly, the currently known efforts to come to a rational choice of drug combination therapy by using primary tumor cell cultures and xenograft studies are confronted by high costs and a variable rate of success in tumor cell growth inhibition, and struggle to obtain highly accurate predictions within the timeframe limited by disease progression. While cancer cell line cultures rarely allow for discoveries that can be directly transferred to a clinical setting, they do allow for experimental investigation of mechanisms underlying biological diversity and robustness and can thus be used to explore strategies to identify potentially effective drug combination therapies. They can therefore contribute to establish a large arsenal of advantageous drug combinations accompanied by prognostic tools enabling the choice of the right combination for the individual tumor. However, even in these cellular models, it is not feasible to test all potential drug combinations and application modes for a sufficient spectrum of cancer cell types.

There is therefore a lot of value in techniques that reduce the experimental search space of drug combinations and their application modes in order to obtain a qualified repertoire of individual drugs and combination therapies for clinical trials, and ultimately to support the delivery of personalized treatment. In this regard, computational models are being increasingly used to predict drug effects, with the aim to rationalize and economize the experimental bottleneck.

There is a need is improve on known models for simulating the effect of drugs, and combinations of drugs, on cancerous cells in order to narrow the experimental search space, More generally, there is a need to provide a technique for improving the determination of drugs, or combinations of drugs, for the treatment of diseased cells.

### SUMMARY OF INVENTION

According to a first aspect of the invention, there is provided a method for simulating a response of a drug, or a combination of drugs, being used tor the treatment of a disease, the method comprising the steps of: generating a model of cell responses in a biological network of cellular processes, wherein the model is a self-contained logical model that comprises a network topology with nodes, edges between nodes and parameters of the nodes for modelling obtained state data or a plurality of biological signalling entities of one or more diseased cells, wherein generating the model comprises determining logical rules that define at least the parameters of the nodes such that an attractor of the model substantially corresponds to said obtained state data of the plurality of biological signalling entities of one or more diseased cells; simulating the effect of a drug, or a combination of drugs, by determining an output of the generated model when the states of one or more nodes of the model are changed in accordance with the expected effect of the drug, or the combination of drugs, on one or more of said biological signalling entities, and determining a drug, or a combination of drugs, for the treatment of the one or more diseased cells in dependence on the output of the model.

Preferably, the method is automatically implemented by a computing device.

Preferably, the biological signalling entities comprise one or more of genes, transcripts, peptides, proteins, protein modification states, small molecules, complexes, metabolites and modifications thereof.

Preferably, the cell responses are cell fate decisions.

Preferably, the computing device generates a plurality of said models of cell responses in a biological network of cellular processes.

Preferably, the generation of each model comprises automatically parameterizing the model to increase the similarity of an attractor of the model with said obtained state data of the plurality of biological signalling entities of one or more diseased cells.

Preferably the method further comprises selecting a plurality of models for use in simulating the effect of a drug, or combination of drugs, in dependence on the fitness of an attractor the selected models being above a threshold level.

Preferably, said logical rules for defining at least the parameters of the nodes are generated by a genetic algorithm.

Preferably, the obtained state data is of one or more diseased cells that are unperturbed.

Preferably, the method further comprises: obtaining data defining the expected effect on one or more signalling entities by each of a plurality of drugs; automatically determining drugs, and/or combinations of drugs, for simulating the effect of; using the one or more models to simulate the effect of the determined drugs and/or combinations drugs; automatically determining drugs, and/or a combinations of one of more of the drugs, for the treatment of the one or more diseased cells in dependence on the outputs of the one or more models

Preferably, the one or more diseased cells are one or more cancerous cells.

Preferably, said logical rules for defining at least the parameters of the nodes are generated by an iterative algorithm.

Preferably, the one or more diseased cells are differentiated cells and the model is generated so that it has a plurality of attractors for modelling the differentiated cells.

Preferably, said state data of a plurality of biological signalling entities of one or more diseased cells is obtained from characterization of a cell or tissue sample and then manually input into the computing device.

According to a second aspect of the invention, there is provided a computing device configured to perform the method of the first aspect.

According to a third aspect of the invention, there is provided a method comprising: analysing one or more diseased cells to obtain state data of a plurality of biological signalling entities of one or more diseased cells; using a computer-implemented method according to the first aspect to determine one or more drugs as candidates for treating the one or more diseased cells; and selecting the determined one or mere drugs and using the selected one or more drugs in practical experimentation in order to determine their effectiveness at treating the disease.

According to a fourth aspect of the invention, there is provided a system for determining a drug, or a combination of drugs, for the treatment of a disease, the system comprising: an analyser for analysing one or more diseased cells to determine the state data of a plurality of biological signalling entities of one or more diseased cells; a computing device according to claim 14 for determining one or more drugs and/or combinations of drugs for treating the diseased cells; and an output to a user that is dependent on the determined one or more drugs and/or combinations of drugs.

### LIST OF FIGURES

Embodiments of the present invention will be described, by way of example only, with reference to the accompanying drawings, in which:

FIG. 1 shows a logical model according to an embodiment;

FIG. 2 is a logical model according to an embodiment that is a reduction of the model in FIG. 1;

FIG. 3 shows simulated effects of combined inhibitors on cell growth;

FIG. 4 shows a process for generating and using one or more models according to an embodiment;

FIG. 5 shows components in an implementation of a model generation process according to an embodiment;

FIG. 6 shows the fitness of a plurality of models according to an embodiment;

FIG. 7 is a table that shows predicted drug synergies by simulations;

FIG. 8 shows measured results of a drug synergy that was determined according to embodiments; and

FIG. 9 is a flowchart of a method according to an embodiment.

### DESCRIPTION

Embodiments of the invention improve on known models for aiding the determination of drug(s) for the treatment of a disease. At least one model of signalling states of one or more diseased cells is generated. The at least one model is a self-contained logical model that has an attractor that substantially corresponds to experimentally determined steady states of signalling entities of the one or more diseased cells. The at least one model is used to simulate the effect of a drug, or a combination of drugs on the one or more diseased cells. Advantageously, a fast and inexpensive technique is provided for determining drugs, or combinations of drugs, for the treatment of the disease.

A cellular structure comprises biological signalling entities. Each signalling entity may be a protein, gene, transcript, metabolite or other property of the cellular structure with a state that can be measured, inferred or estimated. In a cellular processes there are interactions between the signalling entities such that the state of a signalling entity may both influence the state of other signalling entities and also be influenced itself by the states of one or more other signalling entities. The states of the signalling entities allow a diseased cellular structure to be characterised. For example, cancerous cells are typically characterised by the signalling entities reaching a steady state that defines cell growth.

According to embodiments, a steady state of the signalling entities of a diseased cellular structure is experimentally determined and one or more models are generated that represent a steady state of the signalling entities of the diseased cellular structure. Preferably, the experimental data is determined from unperturbed cells so that the model of unperturbed cells does not require large amounts of experimental drug perturbation data.

Each model is a logical network comprising nodes and edges. A node represents a biological signalling entity of a cellular structure. The edges, also referred to herein as interactions, are connections (represented as lines) between nodes in the model and each edge represents how the state, or value, of a node is dependent on the state, or value, of another node in the network.

The state of each node in a model is defined by a logical function that has as inputs the states of the one of more nodes in the model that are linked to the node by edges. The logical function of a node may be a Boolean function and is referred to herein as the parameter of the node. A target node is a node whose state is determined by a logical function, such as a Boolean equation. A target node has incoming interactions from a set of nodes in a graph. A regulator node is a node in a logical function, such as a Boolean equation, that influences the state of a target node. A self-contained model is a model in which all regulator nodes are also target nodes. The outputs of the model, however, are only targets. Each model according to embodiments is self-contained in that all of the nodes are only defined by other nodes in the model and not any external influences. The effect of this is that the cellular process being modelled is not modelled with the effect of external influences, such as a response to growth hormone, unless the state of said growth hormone is regulated by the disease model, i.e. the growth hormone is also a target node.

The mathematical construct of such state based self-contained logical models is known and its properties are well understood in computer science. The current state of each node defines the subsequent state of each node. A model comprises attractors. An attractor of the model is a particular state of the nodes that causes a subsequent state of the nodes to be the same as said particular state of the nodes. For a simple, or point, attractor, the subsequent state of the nodes is unchanged from the present state of the nodes. For a complex attractor, one or more other states occur before the original state is returned to and there is a cycle of states.

The model comprises one or more output nodes. Each output node is defined so that the state of its output represents known effects determining the fate of the cellular process given particular values of the signalling entities. For example, an output may provide an indication of whether or not growth of the cellular process is expected or if apoptesis, i.e. cell death, can be expected.

In a particularly preferred embodiment, one or more models are generated that have a simple attractor in which the states of the nodes of the model exactly, or substantially, correspond to experimentally determined states of signalling entities. However, embodiments also include models with complex attractors for modelling cell cycle processes.

There are drugs that are known to have the property of targeting signalling entities in cellular processes. To model the effect of such a drug, or a combination of such drugs, on the cellular process, values of nodes in the model are changed in accordance with how the drug, or the combination of drugs, would be expected to act on the signalling entities.

Advantageously, determining drugs, or combinations of drugs, for practical experimentation in accordance with the most promising simulations of drugs, or combinations of drugs, enables a substantial reduction of the number of drugs that need to be tested in order to determine an appropriate drug treatment for diseased cells.

The present inventors have devised a single model, in accordance with the above described mathematical properties, that can be used for drug simulation. The model is described in the publication by the present inventors ‘Discovery of Drug Synergies in Gastric Cancer Cells Predicted by Logical Modeling’, PLOS Computational Biology, 11 (8), e1004426. http://doi.org/10.1371/journal.pcbi,1004426 (viewed on 7 October 2015). A. Flobak el al, that is referred to herein as the Flobak paper. The entire contents of the Flobak paper are incorporated herein by reference. The Flobak paper is a source of the information provided in the background section of the present document and discloses a specific technique for improving on the known techniques discussed in the background section.

The Flobak paper demonstrates that simulations by such a model allow effective combinations of drugs to be determined. The model relies only on experimental observations of baseline characteristics to predict synergies. The model avoids relying on perturbation data to predict synergies as this would require a large experimental search space.

A description of some of the characteristics of the model in the Flobak paper are provided below.

The Flobak paper describes a self-contained Boolean model representing cell fate decisions of gastric adenocarcinoma AGS cells that was developed to predict synergistic drug combinations. Interactions, i.e. the edges between nodes, were obtained from manual curation of scientific publications until a self-contained model encompassing all seven drug target nodes was obtained. The self-contained property results in the network state not depending on externally controlled states. The model encompasses 77 nodes representing signalling entities and phenotypic readout nodes, and 149 interactions. Logical rules for defining the state of a node in the model, i.e. a target, are defined using the default equation:

Target=(A or B or C) and not (D or E or F)

The activating regulators A, B and C of a target are combined with logical ‘or’ operators, and inhibitory regulators D, E and F are combined with ‘and not’ operators. That is to say, any activator can fully activate the target node in the absence of inhibitory activity. Furthermore, the action of any inhibitory regulator can fully inhibit the target, even in the presence of activating input from one or more activators. Most of the nodes in the model were represented by a Boolean variable and could take the binary values ‘0’ or ‘1’. Some of the nodes were associated with discrete but non-binary multileveled variables. These were output nodes. Prosurvival and Antisurvival, that could each take the four values (0, 1, 2, 3) and upstream nodes to these Caspase 3/7 and CCND1 that could take values (0, 1, 2). The non-binary multilevel variable nodes were only used for generating outputs of the model and enabled graded growth promoting/inhibitory effects to be simulated. The global output of the model was the difference between the states of the Prosurvival and Antisurvival nodes.

A subset of equations were manually refined either due to biological reasoning, or due to inconsistencies between model behaviour and experimental observations. A literature review resulted in 21 of 75 signalling entities being annotated as ‘active’ or ‘inactive’. Specifically, this steady state signalling pattern of proliferating AGS was compared with attractors of the model, and the model was refined so that a single stable state was obtained that exactly corresponded with the steady state signalling observations.

The manually derived model definition was used to run logical simulations that predicted five drug synergies among 21 drug combinations, of which four were true positives. 20 of 21 drug combination responses were correctly classified.

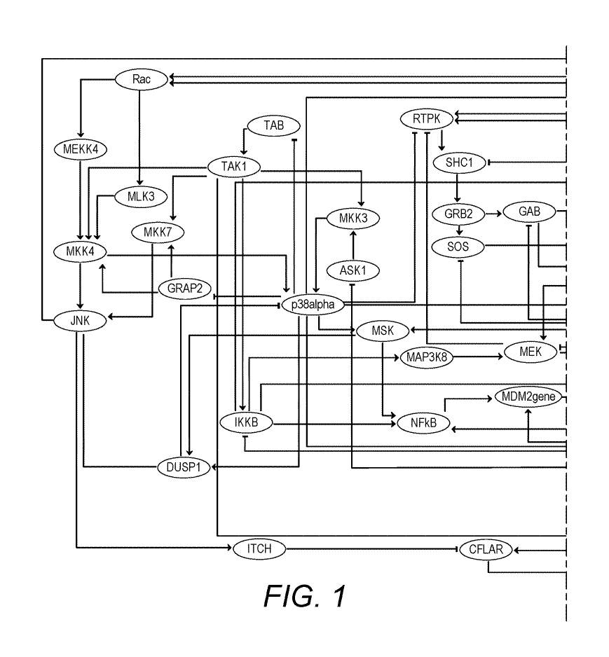

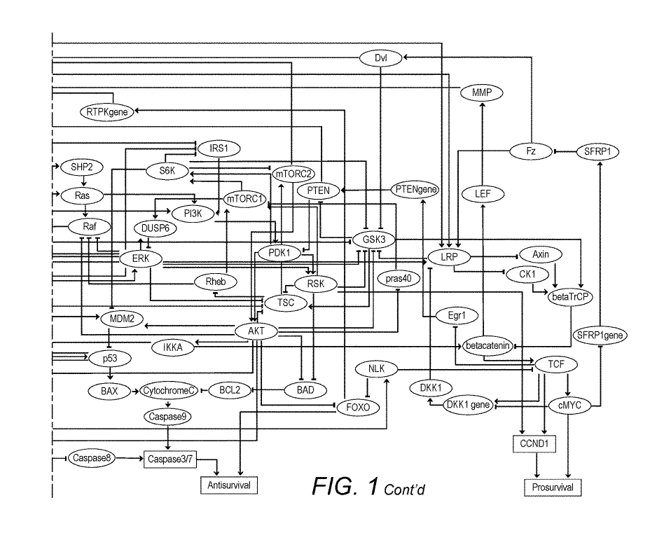

FIG. 1 shows a logical model representing the cell fate decision network governing growth of AGS gastric, adenocarcinoma. The model construction is described in more detail in the Flobak paper.

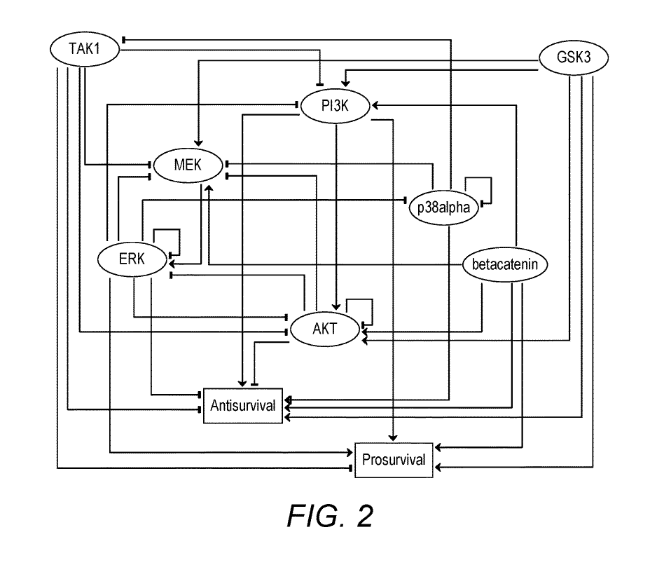

FIG. 2 shows a reduced logical model obtained by reduction of the comprehensive model in FIG. 1. The model reduction is described in more detail in the Flobak paper.

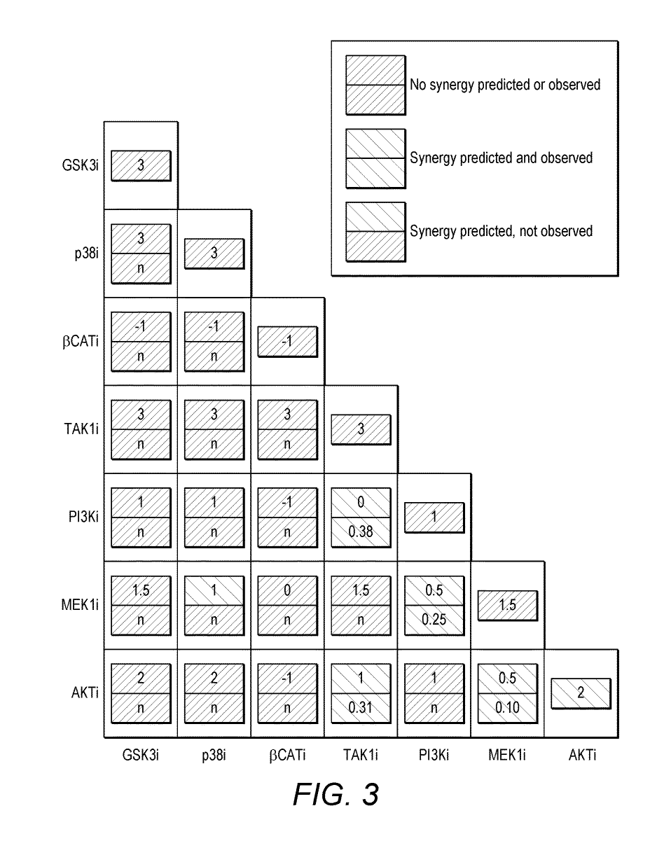

FIG. 3 shows the simulated effects of combined inhibitors on cell growth. It is shown that synergy is simulated by the combined use of, for example, PI3Ki and TAK1i. PI3Ki results in a growth of ‘1’. TAK1i results in a growth of ‘3’. The combined use of PI3Ki and TAK1i results in a lower growth of ‘0’. The simulated effects are described in more detail in the Flobak paper alongside experimentally confirmed results of the simulated synergies.

The Flobak paper demonstrates how a logical model built from known signal transduction network information can be tailored to a specific cancer cell system using baseline data, so that it can be used to predict synergistic and non-synergistic combinatorial growth-impeding treatments. Four of the five predicted synergistic combinations were confirmed experimentally with no false negative predictions. With such a success rate, it would have been sufficient to test less than a quarter of the 21 possible drug combinations investigated and still not miss any synergistic pair.

Contrary to machine learning strategies, which commonly use correlation analysis of large-scale datasets from different disease phenotypes, or large-scale cell culture drug perturbation data to train models for drug response predictions, the modelling strategy in the Flobak paper exploits mechanistic molecular pathway knowledge, available in databases, along with baseline data from the unperturbed cancer cells of the chosen experimental system. This means that the approach allows for the selection of promising candidates for efficient drug combinations before performing actual drug perturbation experiments. The inventors believe that the Flobak paper is the first publication of the advantageous type of model disclosed therein being used in the specific application of drug candidate selection for the treatment of diseased cells.

The majority of regulatory network modelling approaches focus on signalling events driven by specific hormone receptors. This applies to studies investigating logical modelling to understand consequences of interfering with specific growth factor signal transduction responses, as well as to quantitative and semi-quantitative modelling approaches used to predict the effect of synergistic signal transduction perturbations. In contrast, the approach in the Flobak paper demonstrates that it is possible to effectively use a model representing a cell fate decision network in actively growing cells without considering explicitly any external growth-promoting stimulus (e.g. growth hormone). Using the attractor of a self-contained model of a proliferating cell as the reference point for drug synergy analysis provides a good proxy for the state of actively growing cancer cells. Cancer cell growth is considered to be driven by a multitude of growth promoting stimuli. Not only is the potential repertoire of these signals substantial, relatively little detail about their signalling mechanisms is known. The model therefore assumes that their effect can be summarised by considering this multitude of signals to provide a context promoting robust growth and any further detail can therefore be dismissed. On this basis a sustained multifactor-driven proliferation is accommodated by employing a self-contained model, where all components included are regulated by other nodes in the model. The configuration of component activities can then be inferred from baseline biomarkers measured in cancerous cells. Together, these model design principles enable the generation of a dynamic model tailored to specific cancer cells, yet not dependent on explicit extracellular input from specific growth promoting agents (e.g. growth hormones) and without the need for initial large-scale inhibitor perturbation data that would be difficult and costly to obtain.

Although there are a number of advantages provided by the techniques disclosed in the Flobak paper, there are a number of limitations of the techniques disclosed therein.

In the Flobak paper, only a single model of AGS cells is generated. The model edges are exclusively determined from scientific publications describing that specific interaction. The parameterization of the model was based on individual protein states. This required biological insight again from public knowledge in scientific publications (e.g. for a protein ‘complex’ with several proteins acting together, AND was used to signify that all proteins must be active for the protein complex to be active). A few parameterizations were also chosen to obtain a state of a protein that matched the inventors' literature review of AGS cells. The stable state of the resulting model exactly matched an experimentally determined steady state signalling pattern found by the inventors literature review. Accordingly, all edges of the model were derived from public knowledge. The parameterization of the model required public knowledge and/or experimentally determined steady state activities for individual signalling entities

The single model of the Flobak paper can only be constructed and parameterised subject to a large amount of scientific literature being available and the creation of a model typically takes about 2-3 months.

Described below are embodiments that improve on the techniques disclosed in the Flobak paper. The embodiments provide a new approach to generating models for use in simulating the effectiveness of drugs, or combinations of drugs. Advantages include fast, easy and inexpensive generation of the models as well as the ability to include heterogeneity in the model of the cellular structure. The advantages over other approaches to modelling a cellular structure that are inherent to the type of model described in the Flobak paper are maintained.

According to embodiments, the generation of a model is not based on a parameterisation of all the individual protein states. Instead, the entire steady state of the signalling entities is used as a goal for an algorithm that improves the parameterisation. The algorithm is preferably a genetic algorithm. However, other algorithmic approaches for improving the parameterisation may be used. The improvement of the parameterisation is performed automatically by the algorithm being executed by a computer.

Genetic algorithms are known in the art of computer science and are an iterative procedure for improving parameters in order to meet a design goal. A plurality of generations of a model are created with each subsequent generations created in dependence on mutations and/or crossovers between existing generations of models. The creation of further generations by a genetic algorithm is halted when a sufficient fitness threshold is reached with the optimisation goal, or when model evolution does not converge to a solution over a specified number of generations.

Each of the generated models is therefore parameterized by an algorithm, preferably a genetic algorithm, and the parameterisation is iteratively refined until the modelled predictions of signalling entity states matches, or substantially matches, those observed experimentally in a biological system at steady-state (e.g. cancer cell lines freely proliferating, or a tumor in situ that is not subjected to perturbations).

The algorithm can be used to automatically parameterise a model that has an attractor whose state exactly matches to that of the signalling entities of the diseased cells being modelled. However, embodiments preferably generate a plurality of different models. Although the plurality of different models may all have an attractor that exactly matches to that of the signalling entities of the diseased cells being modelled, some or all of the models are preferably similar to the signalling entities of the diseased cells being modelled. For example, the iterations for generating of each model are stopped when an attractor of each model has a 90% fit to the states of the signalling entities of the diseased cells. Advantageously, use of a plurality of different models with variations in the parameterisations result in the effect of heterogeneity within the diseased cells being modelled. That is to say, genetic variations among the diseased cells is accounted for by the model. It is well known for many cancers to have genetic variations across similar cells. The determination of drugs, or combinations of drugs, for treating diseased cells is therefore preferably based on simulation from a plurality of different models rather than a single model as in the Flobak paper.

The interactions of each model may be determined in accordance with known scientific knowledge as in the Flobak paper. However, embodiments also include the use of algorithms, such as genetic algorithms, to modify the interactions in the models. Initial models tor improvement by the algorithm may also be only partially based on interactions in known scientific knowledge, or not based on interactions in known scientific knowledge, and the models can therefore be quickly created even if complete scientific knowledge is not available.

Accordingly, embodiments provide a new approach for generating and using models for determining drugs, or drug combinations, for treating diseased cells.

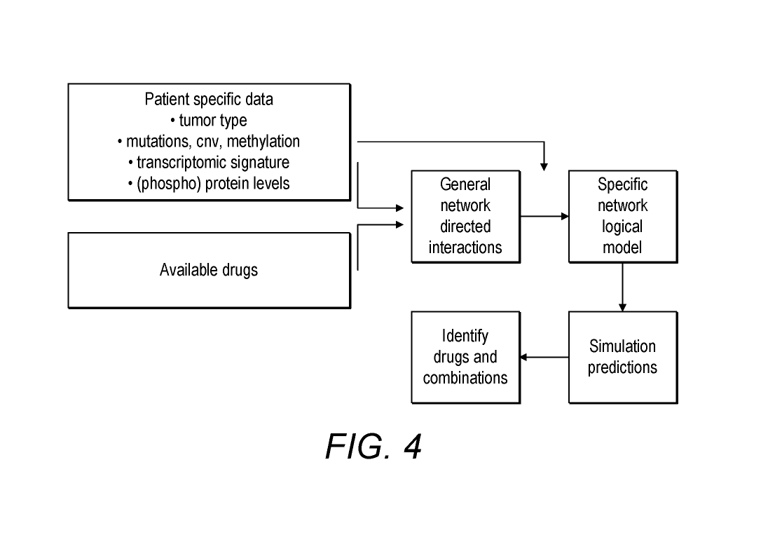

FIG. 4 shows a process for generating and using one or more models for simulating the effectiveness of drugs or combinations of drugs based on patient specific data.

At the start of the process, data is obtained that comprises patient specific data and data on the known properties of available drugs. The patient specific data is obtained from known techniques for analysing diseased cells. The patient specific data may include data on a tumor type, mutations, copy number variation (cnv), methylation, transcriptomic signature, (phospho)-protein levels, etc. The patient specific data is used to determine the biological signalling entities, and the states of the biological signalling entities, to be modelled. The biological signalling entities may comprise one or more of genes, transcripts, peptides, proteins, protein modification states, small molecules, complexes, metabolites and modifications thereof.

Also obtained is data on known properties of available drugs on the market as well as drugs that are currently under development. All of the drugs on which such data is obtained can be considered to be candidate drugs. The candidate drugs that information is obtained for are drugs that are known, or expected, to influence the state of one or more of the signalling entities that have been determined.

A general network is then constructed that is a model comprising nodes representing signalling entitles and edges representing interactions between the signalling entities. The network may be constructed in accordance with the known scientific knowledge as in the Flobak paper. However, embodiments also include experimental determination of the interactions in the models by, for example, genetic algorithms. A model may also be only partially based on interactions in known scientific knowledge, or not based on interactions in known scientific knowledge, and the model can therefore be quickly created even if complete scientific knowledge is not available. The general network can therefore be a single model or a plurality of models.

The general network is then parameterised to generate a specific network. In the specific network an algorithm, such as a genetic algorithm, is used to improve the parameterisation of the model so that an attractor of the model exactly corresponds, or corresponds with a fitness above a threshold, to the states of the signalling entities in the patient specific data.

The improvement of the parameterisation by an algorithm includes changing the simulated effect of activating regulators and inhibiting regulators by automatically modifying the functions that define a target nodes. The modification includes exchanging ‘and not’ definitions with ‘or not’ definitions, and vice-versa. For example:

Target=(A or B or C) and not (D or E or F)

May be automatically changed to:

Target=(A or B or C) or not (D or E or F)

The changes can also be from ‘and’ to ‘or’, and vice-versa, within expressions, for example:

Target=(A or B or C) and not (D or E or F)

May be automatically changed to:

Target=(A and B and C) and not (D or E or F)

For each model provided by the general network, the specific network preferably generates a plurality of different models with each model either exactly corresponding, or corresponding with a fitness above a threshold, to the states of the signalling entities in the patient specific data.

The models are then used to simulate the effects of individual drugs and combinations of drugs.

To simulate the effect of a drug, or combination of drugs, the states of signalling entitles in the model can be fixed. For example;

Target=(A or B or C) and not (D or E or F)

Could be changed by a drug that inhibited that signalling entity to:

Target=(i.e. Target=FALSE)

For drugs with the opposite effect on the target, the state of the target would instead be changed to ‘1’, i.e. TRUE.

The resulting change in the values of the output nodes of the model simulate the effect of the drug.

Each model may be subjected to simulations for discovering potential drug synergies, using a stable state search algorithm, such as that described Veliz-Cuba, A., Aguilar, B., Hinkelmann, F., & Laubenbacher, R. (2014). Steady state analysis of Boolean molecular network models via model reduction and computational algebra. BMC Bioinformatics, 15, 221. http://doi.org/10.1186/1471-2105-15-221.

The simulation therefore automatically identifies promising drugs and combinations of drugs for practical experimentation and/or for the treatment of a disease.

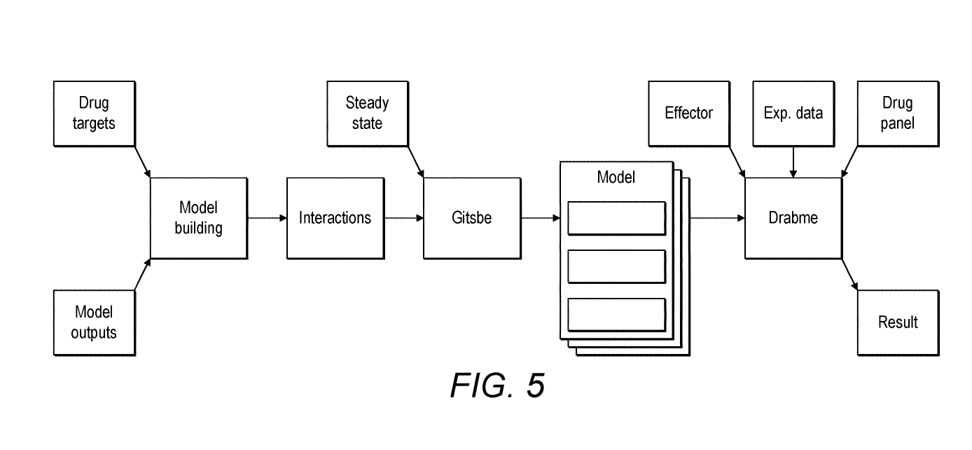

FIG. 5 shows components in an implementation of the model generation process according to an embodiment.

There is a three step pipeline.

In a first step of the pipeline, a model is automatically constructed from a set of drug targets, represented by model nodes, as well as model output nodes used for analysis (such as caspase activation).

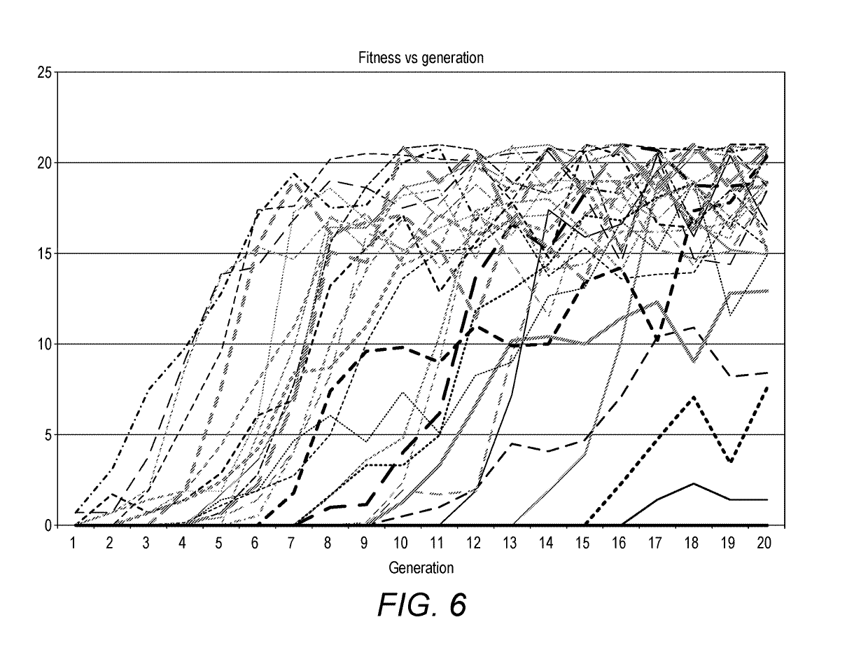

The resulting network of nodes and interactions between the nodes is passed to the second step of the pipeline, referred to as Gitsbe. Gitsbe parameterises the model using genetic algorithms, and where fitness is determined with respect to an experimentally observed steady state of the modelled cells, i.e. a biomarker characterization of the modelled cells. FIG. 6 shows the fitness of a plurality of models increasing as they are iteratively refined by Gitsbe. An ensemble of models is generated by selecting the models with the highest fitness.

The ensemble of models is then passed to a drug response module, referred to as Drabme, in a third step of the pipeline. In the Drabme module, model output nodes (effectors) each are assigned a weight on global output (growth), and a drug panel with drug targets is specified. Optionally experimental data is also obtained so that comparisons can be made. The output result from the Drabme module are the effects of each individual drug and of drug combinations. If experimental data has been obtained, correlations between the simulated and measured data may also be reported.

In order to predict growth of individual cell lines a set of output nodes of each model are defined. Each output node has a weighted and signed score determining the influence on cell fate. Cellular signalling nodes are loosely grouped as either regulators or effectors, where regulators represent signalling proteins that together dictate the state of effectors, and effectors represent proteins that carry out vital cellular functions ultimately deciding cell fate (cell growth, apoptosis etc). Some effector proteins may also regulate the state of regulators via feedback loops.

Effectors are assembled in output nodes of each model, where the summation of states of effector nodes allows simulations to result in either a prosurvival or an antisurvival signalling context. For each effector node a weight is assigned to allow some effectors to have a stronger impact on overall output than others (i.e. CASP8 has a stronger impact on antisurvival signalling than TP53).

The following inclusion criteria can be used for output nodes:
a) the protein is regulated by the signal transduction pathways modelled using information from a reference database used for automated model building; and
b) the protein is directly involved in, or represents a ‘point of no return’ to, execution of a cellular function ultimately deciding cell fate (cell growth, apoptosis etc.)

At least three sources can be used to retrieve effectors for modelling:
1) Manually curated output nodes as used in the Flobak paper. These have already proven to be suitable output nodes in logical model simulations and prediction of cell fate.
2) From driver genes listed in Intogen (Rubio-Perez et al, In silico prescription of anticancer drugs to cohorts of 28 tumor types reveals targeting opportunities, Cancer Cell. 2015 Mar. 9; 27 (3):382-86, PMID: 25501392; Schroeder et al, OncodriveROLE classifies cancer driver genes in loss of function and activating mode of action., Bioinformatics, 2014 Sep. 1; 30 (17):i549-55. doi: 10.1093/bioinformatics/btu467., PMID: 25161246), Cancer Gene Concensus (Futeral et al, Futreal P A, Coin L, Marshall M, Down T, Hubbard T, Wooster R, Rahman N, Stratton M R. A census of human cancer genes, Nat Rev Cancer. 2004 March; 4 (3):177-83, Review, PMID: 14993899) and TSGene (Zhao et al, TSGene: a web resource for tumor suppressor genes, Nucleic Acids Res. 2013 January; 41 (Database issue):D970-6, doi; 10.1093/nar/gks937. Epub 2012 Oct. 12, PMID: 23066107). Driver genes can be broadly grouped into oncogenes that drive tumorigenesis by promoting cell survival and metastasis and tumor suppressor genes that prevent tumourigenesis.
3) From proteins included in RPPA analyses.

Other sources of effectors are also possible and embodiments include models generated with some, or all, of the above-listed sources of effectors not being used.

Selected proteins can be annotated according to the processes they regulate (apoptosis, cell cycle, etc.) based on publicly available information.

In an embodiment, automatic parameterisation optimisation using a genetic algorithm was used to generate an ensemble of 150 logical models. A condition on each model was that it had a fitness, or similarity, of 90% or more to the observed steady state signalling activities in the diseased cells under test. Logical simulations of all single and combinatorial perturbations identified 10 potential synergies among 21 drug combinations, of which 4 are known true positive drug synergies and no false negative predictions were made. The model ensemble was obtained automatically in less than an hour by a standard desktop computer. Clearly the time required to generate the model ensemble could be significantly reduced if more advanced computing means were used, such as a computing cluster or super computer. In contrast, the single model in the Flobak paper had to be manually derived and took months to obtain.

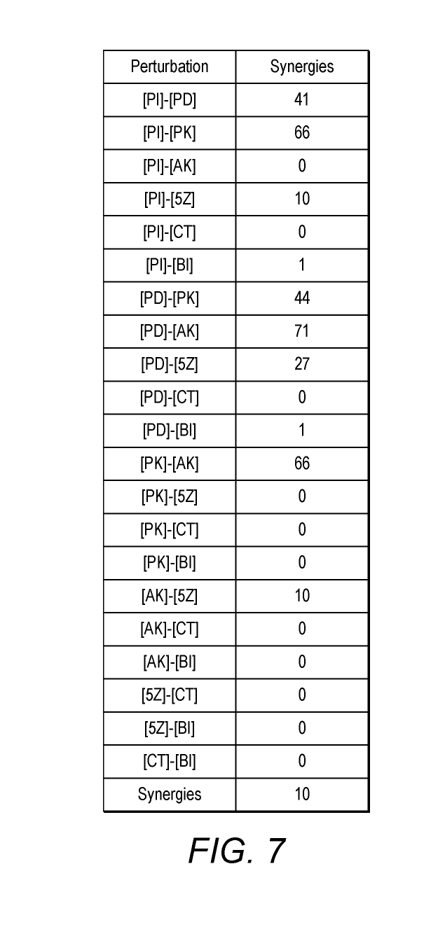

FIG. 7 is a table that shows the drug synergies that were predicted from a drug panel of seven individual drugs and 21 pairwise combinations. The overall impact on predicted growth for each drug combination was simulated and compared to the impact of each drug individually. Whenever the simulated use of two drugs in combination with each other increased the impact from the drugs being used individually, synergy was determined.

The second column of the table in FIG. 7 indicates the number of models (of 150) that predicted synergy. If the cutoff is set at 10 models, these simulated results indicate that eight drug combinations are predicted to act synergistically. Advantageously, the experimental search space is reduced to less than half of the original search space.

Experimental determination of drug synergy was performed as in the Flobak paper. Synergies that were confirmed by practical experimentation were: PI-PD, PI-5Z, PD-AK and AK-5Z.

To validate the beneficial response to one of the combination predicted and validated in cell line experiments the inventors generated subcutaneously injected xenograft tumors in Balb/c mice. 100 μl of 2×105 AGS cells were mixed with 100 μl of Matrigel and injected subcutaneously n the right flanks of 40 mice. Tumors were allowed to grow for 4 weeks, and mice with palpable tumors (n=30) were randomized to four groups: control, PI103 (5 mg/kg/d), (5Z)-7-oxozeaenol (3 mg/kg/d) and combination group which received both PI103 (5 mg/kg/d) and (5Z)-7oxozeaenol (3 mg/kg/d). Mice were injected with inhibitors or vehicle three times per week for a total of seven injections. Tumors were measured twice weekly using callipers and estimated volume from the formula V=0.5*d12*d2, where d1 is shortest diameter and d2 is longest diameter of tumor. Randomization was set up so that tumor volume was similar among groups at onset of treatment. After one week differences in tumor sizes start to display (non-significant), and after 16 days of treatment t-tests show that the combination group displayed both absolute and delta tumor sizes that were significantly smaller compared to all other groups (for absolute sizes p-value 0.023 against (5Z)-7-oxozeaenol alone and p-value 0.027 against PI103 alone, for delta tumor sizes the p-values were 0.012 against (5Z)-7-oxozeaenol alone, and 0.0049 against PI103 alone). Similar results were obtained for Mann-Whitney tests. In contrast, individual agents displayed non-significant activities compared to control, probably due to the low doses used.

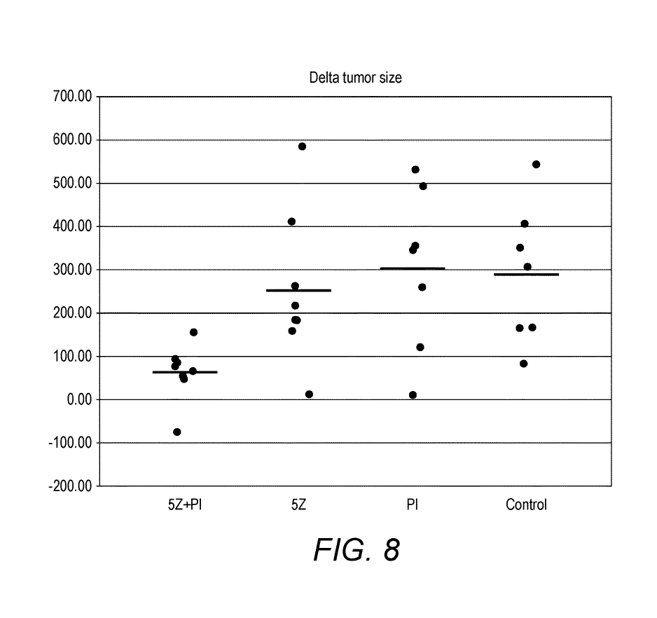

The measured results are shown in FIG. 8.

A number of advantages are therefore provided by the models generated according to embodiments. Models according to embodiments are characterised by:
1) The model being self-contained
2) Logical parameterization of the model
3) The use of genetic algorithms

Advantageously, the models are not dependent on initial perturbation screens. The design principles of the analysis are guided by the premise that growth of cancerous cells is largely driven by mechanisms which enable these cells to exploit a wide range of growth promoting signals from the environment. This aspect of intrinsic, sustained multifactor-driven cancer proliferation is accommodated by constructing the regulatory network as a self-contained model, i.e. the model only includes nodes that are regulated by other nodes in the model. The design obviates the need to model effects of specific growth factor receptors, considering instead the integrated responses from a multitude of growth promoting stimuli, as observed when assessing the activity of signalling entities (proteins and genes) included in the model. A self-contained model can display homeostatic properties in terms of asymptotic behaviour, i.e a system that always seeks the same (set of) attractor(s). It follows from this that the de facto growth promoting configuration of such a self-contained model can be established by observing baseline biomarkers measured in the cancer cells.

The use of models according to embodiments is a significant aid to the prediction of effective drug treatments, in particular the use of drug combination therapy. Advantages include the ability to analyse a sample of diseased cells and, soon after signalling entities have been determined from the analysis, obtaining a model, and simulations by the model, that narrow the search space for effective drug treatment. Such high throughput screening allows personalized treatment.

Embodiments include generating a network topology that is defined by drug targets investigated and biomarkers from patients. Using genetic algorithms, specific models describing cancer signalling can be used for predicting drug responses, including drug resistance and beneficial drug combinations.

Embodiments also include a system comprising an automatic analyser of biological material. The analyser is configured to determine the signalling entities, and states of the signalling entities, of the biological material. The system comprises computing means for automatically generating a model in accordance with the embodiments described herein and using the model to identify drug candidates, or combinations of drug candidates, for the treatment of a disease present in the biological sample. The system also comprises a display on which the identified drug candidates, or combinations of drug candidates, can be displayed.

Some of the above-described embodiments are described with references to algorithms, flowcharts and/or block diagrams of methods, apparatuses, and systems. One skilled in the art will appreciate that each algorithm, block of the flowcharts, block diagrams, and/or their combinations can be implemented by computer program instructions. These computer program instructions may be provided to a processor of a general purpose computer(s) or computer system(s), special purpose computer(s) or computer system(s), other programmable data processing apparatus, or the like, to produce a machine, such that the instructions, executed via the processor of the computer (computer system, programmable data processing apparatus, or the like), create mechanisms for implementing the functions specified by the algorithm and/or within the blocks of the flowcharts and/or block diagrams and/or within corresponding portions of the present disclosure.

These computer program instructions may also be stored in a computer-readable memory (or medium) and direct a computer (computer system, programmable data processing apparatus, or the like) to function in a particular manner, such that the instructions stored in the computer readable memory or medium produce an article of manufacture including instruction means which implement the functions specified in the blocks of the flowchart(s) and/or block diagram(s) and/or within corresponding portions of the present disclosure.

One skilled in the art will understand that any suitable computer-readable medium may be utilized. In particular, the computer-readable medium may include, but is not limited to, a non-transitory computer-readable medium, such as a tangible electronic, magnetic, optical, electromagnetic, infrared, and/or semiconductor system, device, and/or other apparatus. For example, in some embodiments, the non-transitory computer-readable medium includes a tangible medium such as a portable computer diskette a hard disk, a random access memory (RAM), a read-only memory (ROM), an erasable programmable read-only memory (HP OM or Flash memory), a compact disc read-only memory (CD-ROM), and/or some other tangible optical and/or magnetic storage device. In other embodiments, the computer-readable medium may be transitory, such as, for example, a propagation signal including computer-executable program code portions embodied therein.

The computer program instructions may also be loaded onto a computer (computer system, other programmable data processing apparatus, or the like) to cause a series of operational steps to be performed on the computer (computer system, other programmable data processing apparatus, or the like) to produce a computer-implemented method or process such that the instructions executed on the computer (computer system, other programmable data processing apparatus, or the like) provide steps for implementing the algorithms and/or functions/acts specified in the flowchart and/or block diagram block(s) and/or within corresponding portions of the present disclosure.

In some embodiments of the present disclosure, the above described methods and/or processes could be performed by a program executing in a programmable, general purpose computer or computer system. Alternative embodiments are implemented in a dedicated or special-purpose computer or computer system in which some or all of the operations, functions, steps, or acts are performed using hardwired logic or firmware.

In a particularly preferred embodiment, the interactions of models are determined based on the determined states of the signalling entities for the analysis of diseased cells and known scientific knowledge. Genetic algorithms, or other parameterisation optimisation methods, are only used for the parameterisation of the model. Preferably, the only changes made during the optimisation by the genetic algorithm is to change the relation between activating regulators and inhibiting regulators from ‘and’ to ‘or’ and vice-versa, and can also be used to modify operators that determine how the influence of activating, or inhibitory, regulators are computed. Genetic algorithms may also be used to remove interactions from models during optimisation.

An embodiment of the invention is the generation of a model with the properties of the models disclosed herein. The use of the model is not restricted to simulating the effect of drug combinations and the model can be used for other applications.

Although the described embodiments have mainly focussed on drug synergy predictions, embodiments include using the same approach to determine single- and combinatorial drug resistance, and to propose additional drugs to include with conventional treatments for disease. Embodiments also include using the model(s) to aid the treatment of other diseases than cancer. Embodiments can aid any molecularly targeted therapy such as anti-inflammatory treatment, anti-arrhythmic treatment, anti-convulsive therapy and others.

Embodiments are not restricted to the generation of models based on a single observed steady state of the signalling entities of cells. Embodiments also include the generation of models with a plurality of defined attractors so that cellular differentiation is modelled. Differentiated cells can be seen as specific model instances with an attractor that recapitulates cell-specific steady states, where a common progenitor cell cap differentiate to several specific cells. Models according to embodiments can thus shed light on perturbations that will direct differentiation to specific cell types.

A number of techniques for determining the states of signalling entities in a biological system are known. Typically, a sample of diseased tissue is removed from a human or animal body and the sample is then analysed, outside of the body, in a laboratory environment. However, the analysed diseased cells could be a cell line (i.e not a tissue sample, grown in vitro), primary cells derived from laboratory animal or human, a sample of metabolites, etc. When a potential drug, or combination of drugs, has been determined according to the embodiments described herein, the actual effect of the drug, or combination of drugs, should then preferably be determined through practical experiments in a laboratory environment prior to the determined drugs, or combination of drugs, being used on a patient. Accordingly, embodiments provide an aid to the research of drug treatments.

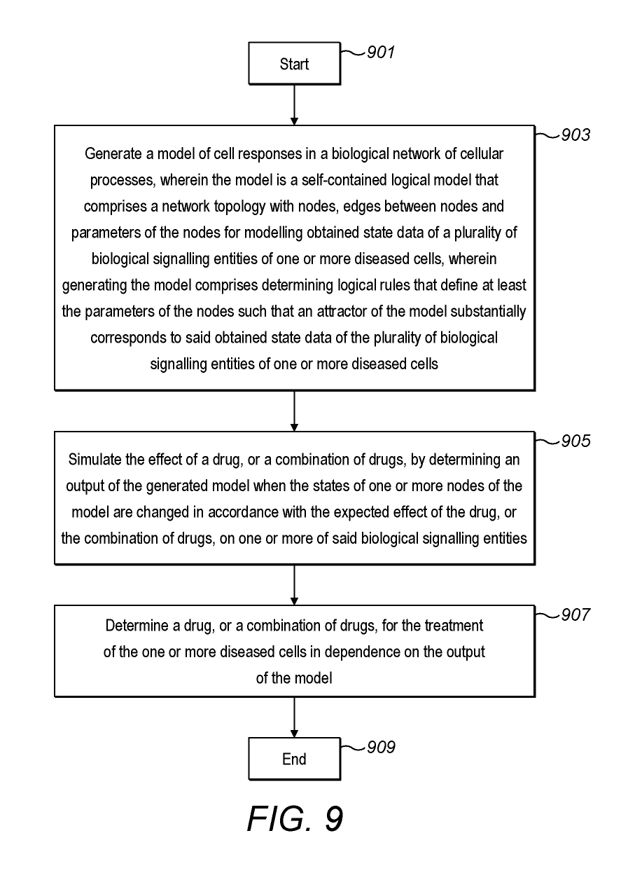

A flow chart of a method according to an embodiment is shown in FIG. 9.

The method starts at step 901.

In step 903, a model of cell responses in a biological network of cellular processes is generated, wherein the model is a self-contained logical model that comprises a network topology with nodes, edges between nodes and parameters of the nodes for modelling obtained state data of a plurality of biological signalling entities of one or more diseased cells, wherein generating the model comprises determining logical rules that define at least the parameters of the nodes such that an attractor of the model substantially corresponds to said obtained state data of the plurality of biological signalling entities of one or more diseased cells.

In step 905 the effect of a drug is simulated, or a combination of drugs, by determining an output of the generated model when the states of one or more nodes of the model are changed In accordance with the expected effect of the drug, or the combination of drugs, on one or more of said biological signalling entities.

In step 907 a drug, or a combination of drugs, is determined for the treatment of the one or more diseased cells in dependence on the output of the model.

In step 909, the process ends.

The flowcharts and description thereof herein should not be understood to prescribe a fixed order of performing the method steps described therein. Rather, the method steps may be performed in any order that is practicable.

Although the present invention has been described in connection with specific exemplary embodiments, it should be understood that various changes, substitutions, and alterations apparent to those skilled in the art can be made to the disclosed embodiments without departing from the spirit and scope of the invention as set forth in the appended claims.

1. A method for simulating a response of a drug, or a combination of drugs, being used for the treatment of a disease, the method comprising the steps of:
generating a model of cell responses in a biological network of cellular processes, wherein the model is a self-contained logical model that comprises a network topology with nodes, edges between nodes and parameters of the nodes for modelling obtained state data of a plurality of biological signalling entities of one or more diseased cells, wherein generating the model comprises determining logical rules that define at least the parameters of the nodes such that an attractor of the model substantially corresponds to said obtained state data of the plurality of biological signalling entities of one or more diseased cells;
simulating the effect of a drug, or a combination of drugs, by determining an output of the generated model when the states of one or more nodes of the model are changed in accordance with the expected effect of the drug, or the combination of drugs, on one or more of said biological signalling entities; and
determining a drug, or a combination of drugs, for the treatment of the one or more diseased cells in dependence on the output of the model.

2. The method according to claim 1, wherein the method is automatically implemented by a computing device.

3. The computer-implemented method according to claim 2, wherein the biological signalling entities comprise one or more of genes, transcripts, peptides, proteins, protein modification states, small molecules, complexes, metabolites and modifications thereof.

4. The computer-implemented method according to claim 2, wherein the cell responses are cell fate decisions.

5. The computer-implemented method according to claims 2, wherein the computing device generates a plurality of said models of cell responses in a biological network of cellular processes.

6. The computer-implemented method according to claim 5, wherein the generation of each model comprises automatically parameterizing the model to increase the similarity of an attractor of the model with said obtained state data of the plurality of biological signalling entities of one or more diseased cells.

7. The computer-implemented method according to claim 6, further comprising selecting a plurality of models for use in simulating the effect of a drug, or combination of drugs, in dependence on the fitness of an attractor the selected models being above a threshold level.

8. The computer-implemented method according to claim 2, wherein said logical rules for defining at least the parameters of the nodes are generated by a genetic algorithm.

9. The computer-implemented method according to claim 2, wherein the obtained state data is of one or more diseased cells that are unperturbed.

10. The computer-implemented method according to claim 2, further comprising:
obtaining data defining the expected effect on one or more signalling entities by each of a plurality of drugs;
automatically determining drugs, and/or combinations of drugs, for simulating the effect of;
using the one or more models to simulate the effect of the determined drugs and/or combinations drugs;
automatically determining drugs, and/or a combinations of one or more of the drugs, for the treatment of the one or more diseased cells in dependence on the outputs of the one or more models.

11. The computer-implemented method according to claim 2, wherein the one or more diseased cells are one or more cancerous cells.

12. The computer-implemented method according to claim 2, wherein said logical rules for defining at least the parameters of the nodes are generated by an iterative algorithm.

13. The computer-implemented method according to claim 2, wherein the one or more diseased cells are differentiated cells and the model is generated so that it has a plurality of attractors for modelling the differentiated cells.

14. The computer-implemented method according to claim 2, wherein said state data of a plurality of biological signalling entities of one or more diseased cells is obtained from characterization of a cell or tissue sample and then manually input into the computing device.

15. A computing device configured to perform the method of claim 2.

16. A method comprising:
analysing one or more diseased cells to obtain state data of a plurality of biological signalling entities of one or more diseased cells;
using a computer-implemented method according to claim 1 to determine one or more drugs as candidates for treating the one or more diseased cells; and
selecting the determined one or more drugs and using the selected one or more drugs in practical experimentation in order to determine their effectiveness at treating the disease.

17. A system for determining a drug, or a combination of drugs, for the treatment of a disease, the system comprising:
an analyser for analysing one or more diseased cells to determine the state data of a plurality of biological signalling entities of one or more diseased cells;
a computing device according to claim 15 for determining one or more drugs and/or combinations of drugs for treating the diseased cells; and
an output to a user that is dependent on the determined one or more drugs and/or combinations of drugs.
